(19) United States
(12) Patent Application Publication
(10) Pub. No.: US 2015/0245946 A1
(43) Pub. Date: Sep. 3, 2015

(54) METHOD OF POSITIONING AN INTRAOCULAR DEVICE

(71) Applicants: The University of Melbourne, Melbourne (AU); National ICT Australia Limited, Eveleigh (AU)

(72) Inventors: Kate Fox, Pascoe Vale South (AU); David Garrett, Braybrook (AU); Kumaravelu Ganesan, Mill Park (AU); Hamish Meffin, Bellfield (AU); Steven Prawer, Caulfield (AU)

(21) Appl. No.: 14/713,998

(22) Filed: May 15, 2015

Related U.S. Application Data
(63) Continuation-in-part of application No. PCT/AU2013/001304, filed on Nov. 12, 2013.
(60) Provisional application No. 61/727,187, filed on Nov. 16, 2012.

(30) Foreign Application Priority Data
Nov. 16, 2012 (AU) 2012905040

(51) Int. Cl. A61F 9/00

(52) U.S. Cl. CPC A61F 9/0017

(57) ABSTRACT

A method of positioning an intraocular device at an intraocular position is provided. The method comprises providing a first and a second element selected such that a magnetic force attracts the first element and the second element to each other. The method also comprises positioning the first element in a suprachoroidal space of an eye and positioning the intraocular device in an intraocular space at a portion of tissue of the eye. The method further comprises positioning the second element in the intraocular space of the eye. The first element, the second element and the intraocular device are positioned such that the portion of the tissue of the eye and at least a portion of the intraocular device are positioned between the first and second elements such that the magnetic force at least contributes to securing the intraocular device at the portion of the tissue of the eye.

### CROSS-REFERENCE TO RELATED APPLICATIONS

Any and all priority claims identified in the Application Data Sheet, or any correction thereto, are hereby incorporated by reference under 37 CFR 1.57. This application is a continuation-in-part of International Patent Application No. PCT/AU2013/001304, filed Nov. 12, 2013 under the Patent Cooperation Treaty (PCT), which was published by the International Bureau in English on May 22, 2014, which designates the United States and claims the benefit of U.S. Provisional Application No. 61/727,187, filed Nov. 16, 2012, and Australian Application No. 2012905040, filed Nov. 16, 2012. Each of the aforementioned applications is incorporated by reference herein in its entirety, and each is hereby expressly made a part of this specification.

### FIELD OF THE INVENTION

The present invention relates to a method of positioning an intraocular device.

### BACKGROUND OF THE INVENTION

A range of medical devices are frequently implanted into the human body. Such devices include pacemakers, ear implants, retinal prostheses and other types of devices.

Damage to the retina of the eye can be caused by degenerative eye conditions and retinal prostheses may be used to electrically stimulate ganglion cells in the inner layers of the retina. Such retinal prostheses are required to be in contact with the retina to stimulate the retinal tissue. However, it is known that the retina is not attached to underlying tissue layers and is merely held in place by the fluid pressure within the eye. Moreover, the retinal tissue is delicate and can be easily damaged by pressure.

Generally, such intraocular devices are secured in a desired position using tacks and the mechanical pressure provided by the vitreous humour. However, tacks cause trauma during insertion, are difficult to handle and cannot be removed without causing significant damage to the eye. Further, tacks do not provide any aid to align the device into a certain position or orientation. Hence, the alignment relies entirely on the manual positioning by the surgeon.

### SUMMARY OF THE INVENTION

The present invention provides in a first aspect a method of positioning an intraocular device at an intraocular position, the method comprising the steps of:
providing a first and a second element, the first and the second element being selected such that a magnetic force attracts the first element and the second element to each other;
positioning the first element in a suprachoroidal space of an eye;
positioning the intraocular device in an intraocular space at a portion of tissue of the eye; and
positioning the second element in the intraocular space of the eye;
wherein the first element, the second element and the intraocular device are positioned such that the portion of the tissue of the eye and at least a portion of the intraocular device are positioned between the first and second elements such that the magnetic force at least contributes to securing the intraocular device at the portion of the tissue of the eye.

The portion of the tissue of the eye may comprise a portion of the retina. The intraocular device may be a retinal prosthesis.

The second element may be coupled to the intraocular device and may be surrounded by, or embedded in, a portion of the intraocular device. Alternatively, the method may comprise coupling the intraocular device to the second element.

The method in accordance with the first aspect of the invention has significant advantages. In this regard, it will be appreciated that there are certain technical factors and difficulties to be considered when employing magnets in retinal prostheses. An attractive force between a pair of magnets is constant. Accordingly, anything that lies between a pair of attracted magnets is under compression, and therefore liable to damage because retinal tissue is delicate and can be easily damaged by pressure. Further, although biological tissue is typically at least partially elastic, there is still generally a risk that the elasticity of tissue may not be sufficient to counter the magnetic force over time, and thus may yield under constant force. For example, it is known that in dental implants that use magnets, a magnet can bore through tissue to reach its mate. On the other hand, the magnetic force ought to be sufficiently powerful to maintain the position of the intraocular device with respect to the retina, and also withstand any other forces that might tend to dislodge the device when in use.

By positioning the first element in a suprachoroidal space of an eye, the present invention provides the advantage that a distance between the first and second elements (being magnetically attracted to each other) is minimised. This in turn allows for the use of a lower power magnetic force, for example compared to external positioning of the first element. Thus, the first and second elements can be chosen so that a magnetic attractive force between the elements can also be minimised to reduce the risk of damage to the retina, yet have sufficient strength to hold the intraocular device in place. In one embodiment, the distance between the first and second elements may be in the vicinity of 0.9-1.3 mm.

Another advantage is that the use of the suprachoroidal space provides the additional benefit of assisting by physically holding the elements in place as compared to an external magnet placement. An external location for a magnet is undesirable since it would require a higher magnetic force or an additional mechanism for maintaining the external magnet in position and avoiding dislocation.

A further advantage is that the step of positioning the first element in the suprachoroidal space of the eye, i.e. between the sclera and the choroid of the eye is often less complicated than positioning the first element at another suitable position. Furthermore, by positioning the first element in the suprachoroidal space movability of the first element is limited, which improves an anchoring function of the first element for the intraocular device.

In some embodiments, the first element is coupled to or embedded in a flexible material portion that is shaped to facilitate insertion and/or positioning of the flexible portion with the second element in the suprachoroidal space of the eye. For example, the flexible portion may comprise a tapered end-portion that facilitates insertion of the flexible portion into the suprachoroidal space of the eye.

The method may also comprise coupling the first element to the flexible portion such that the first element is attached to the flexible portion or partially surrounded by the flexible portion. Coupling the first element to a flexible portion may also comprise embedding the first element in the flexible portion and may further comprise hermetically sealing the flexible portion.

The flexible portion typically comprises a biocompatible polymeric material that is sufficiently flexible to conform to a curvature of suprachoroidal space. In one specific embodiment, the flexible portion comprises a suitable polymeric material, such as silicone that may be reinforced using for example suitable fibres.

In a first embodiment the first and second elements are selected and positioned such that the intraocular device is secured at a desired position without the need for additional fasteners. This embodiment provides the advantage that fasteners (such as tacks) and related trauma may be avoided.

In an alternative second embodiment the first and second elements are arranged to position the intraocular device in a desired position and the method comprises securing the intraocular device subsequently in the desired position using suitable fasteners such as tacks. In the second embodiment the first and second elements may be selected such that the magnetic forces are weaker than in the first embodiment, which provides the advantage that an impact on tissue of the eye such as the retina and the choroid by the positioned intraocular device (and caused by the magnetic forces) is reduced.

In one embodiment the method comprises positioning the intraocular device in a predetermined angular orientation relative to the retina using the magnetic force. In this case the first and second elements may comprise materials that have magnetic properties that are arranged such that the magnetic force is directed to position the first and second elements in the predetermined angular orientation relative to each other. The second element may be coupled to a portion of the intraocular device and the first element may be anchored in the suprachorodial space and the method may comprise positioning the second element with the intraocular device in the predetermined angular orientation relative to the retina using the magnetic force.

In the above-mentioned embodiment both the first and second elements may comprise permanent magnetic materials. At least one of the first and second elements may also comprise two or more magnetic materials that are positioned within the respective first or second element such that the magnetic force is directed to position the first and second elements relative to each other in the predetermined angular orientation.

The method may comprise forming an incision through the sclera of the eye such that the first element or a flexible portion comprising the first element can be inserted into the suprachoroidal space. The method may also comprise moving the sclera and the choroid from each other by inserting the flexible portion with the first element into the suprachoroidal space.

The method may comprise heat treating at least one of the first and second elements prior to positioning the first and second elements, in order to reduce a magnetic strength between the first and second elements from an original strength prior to heat treatment.

The present invention provides in a second aspect an intraocular system, comprising:
an intraocular device for positioning in an intraocular space of an eye;
a flexible portion arranged for locating in the suprachoroidal space of the eye;
a first element coupled to, or surrounded by, the flexible portion;
a second element arranged for positioning in the intraocular space of the eye;
wherein the first element and the second element are arranged such that a magnetic force attracts the first element and the second element to each other, and wherein the first element, the second element and the intraocular device are arranged such that, when the intraocular device is positioned, a portion of tissue of the eye and at least a portion of the intraocular device are positioned between the first and second elements such that the magnetic force at least contributes to securing the intraocular device at the tissue of the eye.

The flexible portion typically comprises a biocompatible polymeric material that is sufficiently flexible to conform to a curvature of suprachoroidal space. In one specific embodiment, the flexible portion comprises a suitable polymeric material, such as silicone that may be reinforced using for example suitable fibres. The flexible portion may also comprise a tapered end-portion that facilitates insertion of the flexible portion into the suprachoroidal space of the eye.

In one embodiment the first element is embedded in the flexible portion.

At least one of the first and the second elements may comprise a permanent magnetic material. At least one of the first and second elements may also comprise one or more magnetic materials that may be positioned within the first and second elements such that the magnetic force positions the first and second elements in the predetermined angular orientation relative to each other.

The intraocular device may be a retinal prosthesis.

The invention will be more fully understood from the following description of specific embodiments of the invention. The description is provided with reference to the accompanying drawings.

### BRIEF DESCRIPTION OF THE DRAWINGS

FIG. 1 is a flow chart illustrating an embodiment of the present invention;

FIG. 2 is a schematic perspective representation of a positioned intraocular device in accordance with an embodiment of the present invention;

FIG. 3 is a schematic cross-sectional representation of a positioned intraocular device in accordance with an embodiment of the present invention;

FIGS. 4(a) and 4(b) show a top and a side view of the flexible portion of the intraocular device of FIG. 3 including the first element; and

FIGS. 5 (a) to 5 (c) illustrate elements having magnetic materials in accordance with embodiments of the present invention.

FIG. 6(a) is a perspective view of an intraocular system in accordance with another embodiment of the present invention.

FIG. 6(b) is a sectional view of an intraocular system shown in use in accordance with an embodiment of the present invention.

FIGS. 7(a) and 7(b) are a side view and top view, respectively, of components of an intraocular system, according to an embodiment of the present invention.

### DETAILED DESCRIPTION OF SPECIFIC EMBODIMENTS

Embodiments of the present invention relate to a method of positioning an intraocular device within a human eye. In one embodiment the intraocular device is an epiretinal device that is positioned on the inner surface of the retina of the eye. However, a person skilled in the art will appreciate that other intraocular devices are envisaged.

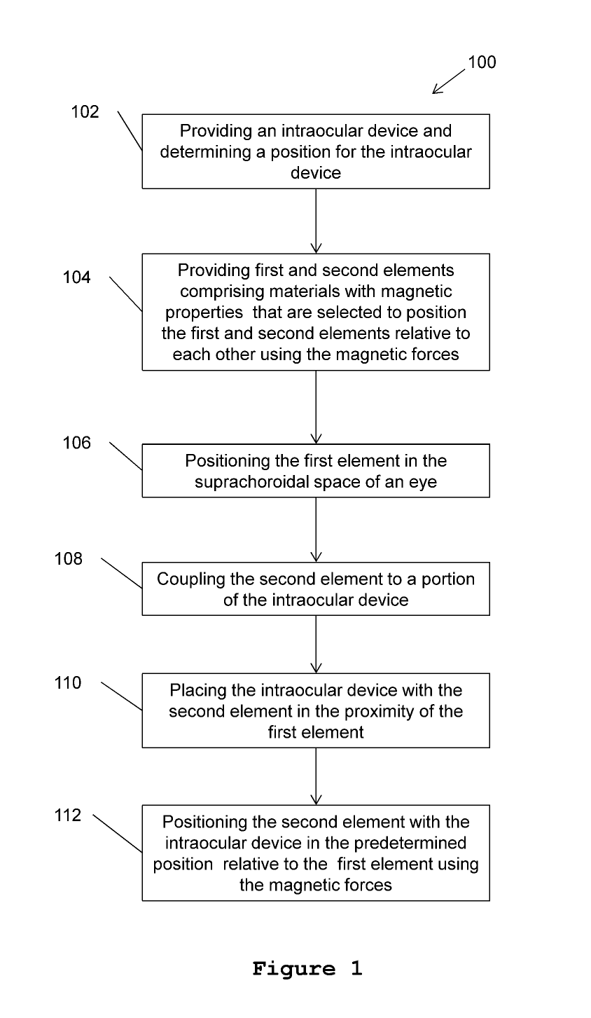

Referring initially to FIG. 1, a method 100 of positioning an intraocular device in accordance with an embodiment of the present invention is described.

The method 100 comprises the initial step 102 of providing the intraocular device and determining a position for the intraocular device within the human eye. The intraocular device is in this example a retinal prosthesis. The position of the intraocular device may, for example, be a location on the inner surface of the retina of the eye or on the outer surface of the retina of the eye.

Step 104 provides first and second elements that comprise permanent magnetic or magnetisable materials. The materials are configured to generate attracting magnetic forces between the first and the second element. In a specific example the first and second elements are also arranged such that the magnetic forces rotate the materials relative to each other in a predetermined angular orientation. The permanent magnetic materials may for example be ferro-magnetic materials that comprise iron, nickel, cobalt, Alnico, iron oxides or rare earth-based materials such that materials that comprise neodymium and samarium-cobalt. The magnetisable materials may also comprise for example iron, nickel, cobalt and rare earth materials such as neodymium and samarium-cobalt. The first and/or the second elements may also be formed from more than one permanent magnetic or magnetisable material. Heat may also be used to modify the degree of magnetisation.

Step 106 comprises positioning the first element in a predetermined position within the suprachoroidal space of the human eye, i.e. between the sclera and the choroid of the human eye. For example, the first element may be positioned with a predetermined angular orientation at a position within the suprachoroidal space. The first element may be secured using any suitable technique and may for example be secured using micro-tacks or glue.

In one embodiment, the first element is at least partially surrounded by a flexible portion. In this embodiment, the method comprises a further step of providing a flexible portion that is arranged for locating at a position within the suprachoroidal space of the human eye. In this example, the flexible portion comprises a silicone material and is sufficiently flexible to conform to the curvature of the suprachoroidal space. Additionally, the method comprises a step of coupling the first element to the flexible portion such that the flexible portion at least partially surrounds the first element. In one example, the first element is embedded in the flexible portion such as hermetically sealed within the flexible portion.

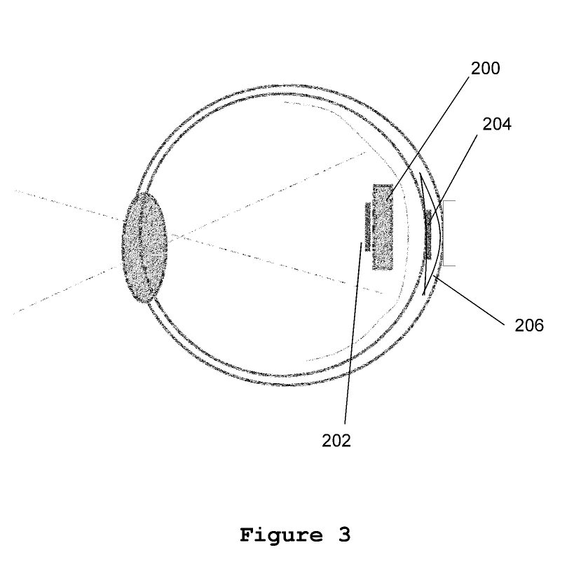

Examples of the flexible portion will further be described with reference to FIG. 3.

By positioning the first element in the suprachoroidal space, the first element may be secured at the predetermined position without the need for additional securing means such as micro-tacks or the like. The first element may be embedded within a flexible portion and the flexible portion may be sandwiched between the choroid and the sclera and thereby sufficiently secured at that position.

Furthermore, positioning the first element in the suprachoroidal space is often less complicated than positioning at another suitable position. An incision is made through the sclera so that the first element can be inserted into the suprachoroidal space. A space between the sclera and the choroid in form of a pocket is formed by a suitable device or by virtue of the shape of the first element or the flexible portion comprising the first element.

Step 108 comprises coupling the second element to a portion of the intraocular device. For example, the second element may be mechanically coupled to the medical device or attached using a suitable biocompatible adhesive. Alternatively, the second element may be embedded in the intraocular device. A person skilled in the art will appreciate that the intraocular device may be coupled to the second element using a variety of techniques.

Step 110 comprises placing the assembly comprising the intraocular device and the second element into the intraocular space and in the proximity of the first element that is positioned in the suprachoroidal space. In this embodiment the assembly comprising the intraocular device and the second element is introduced into the human eye and placed in proximity of the inner surface of the retina in the proximity of the first element, such that a portion of the intraocular device and a portion of the tissue of the eye is positioned between the first and second elements.

Step 112 comprises positioning the assembly comprising the intraocular device and the second element in the predetermined position and in a predetermined angular orientation relative to the first element using the magnetic forces between the first and second elements.

In one embodiment of the present invention the materials are selected such that the magnetic forces are sufficient to hold the medical device in position without further intervention. The flexible portion is shaped to conform to the curvature of the suprachoroidal space and is secured in the suprachoroidal space. In other words, the flexible portion is sandwiched between the choroid and the sclera. In this way, there is no need for further securing means or manipulations.

In an alternative embodiment, the flexible portion is coupled to the first element and is secured to a portion of the sclera and/or the choroid by virtue of securing means. Suitable securing means include micro-tacks, sutures, and adhesive. Whilst the flexible portion is secured by a suitable securing element, the second element coupled to the intraocular device may be held at the position at the inner surface of the retina only by the magnetic forces between the first and second elements.

In a further alternative embodiment, the first and second elements are designed such that the magnetic forces are weaker and the application of further securing means (such as micro-tacks) or manipulations may be required.

Referring back to the method 100 of positioning the intraocular device, the method 100 may further comprise surgical procedure steps in relation to implanting the intraocular device in a human eye. Exemplary surgical procedure steps may include forming an incision through the sclera of the eye using a scalpel such that the flexible portion with the first element can be inserted into a space between the sclera and the choroid. The incision typically is slightly wider than a width of the flexible portion.

A space in form of a pocket within the suprachoroidal space may be formed by using the shape of the flexible portion or any other suitable device such that the flexible portion with the first element can be positioned in the formed pocket. Once the flexible portion is fully inserted and positioned within the suprachoroidal space, the incision may for example be closed using suitable sutures.

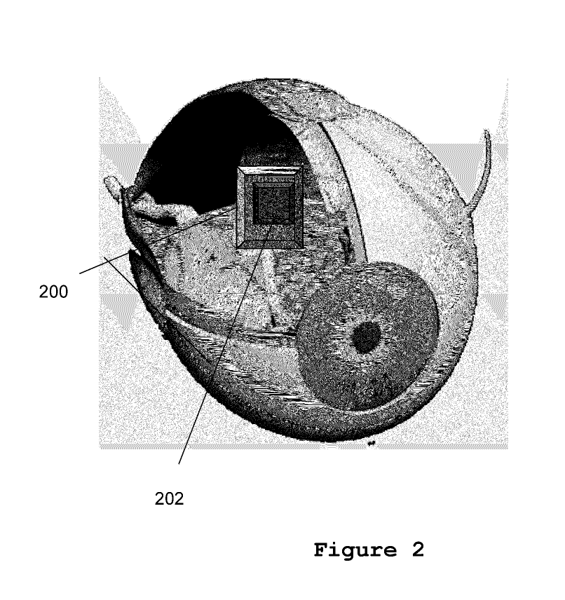

FIGS. 2 and 3 are schematic illustrations of a positioned intraocular device 200 in accordance with an embodiment of the present invention. In this specific embodiment the intraocular device 200 is placed on an inner surface of the retina of the eye and is held in position by the second element 202 and the first element 204 located within the suprachoroidal space of the eye between the choroid and the sclera of the eye. In this example, the first and second elements 202, 204 are positioned such that a portion of the intraocular device 200, a portion of the retina and a portion of the choroid are located between the first and second elements 202, 204. As a consequence of the attracting magnetic forces between the first and second elements 202, 204, the intraocular device can be positioned and secured at the inner surface of the retina. The tissue on the outer surface of the eye is less delicate than the tissue on the inner surface of the eye, such as the retina of the eye.

In this particular embodiment, the first element 204 that is positioned in the suprachoroidal space is embedded in a flexible portion 206 such as a silicone portion. However, the flexible portion may comprise any other suitable biocompatible polymeric materials. The flexible portion 206 is arranged to locate (and secure) the first element 204 in the suprachoroidal space. Specifically, the flexible portion 206 is sufficiently flexible to conform to the curvature of the sclera and the choroid thereby securing its position in the suprachoroidal space.

In addition, by providing a portion that is sufficiently flexible, an impact on the surrounding tissue of the eye can be reduced.

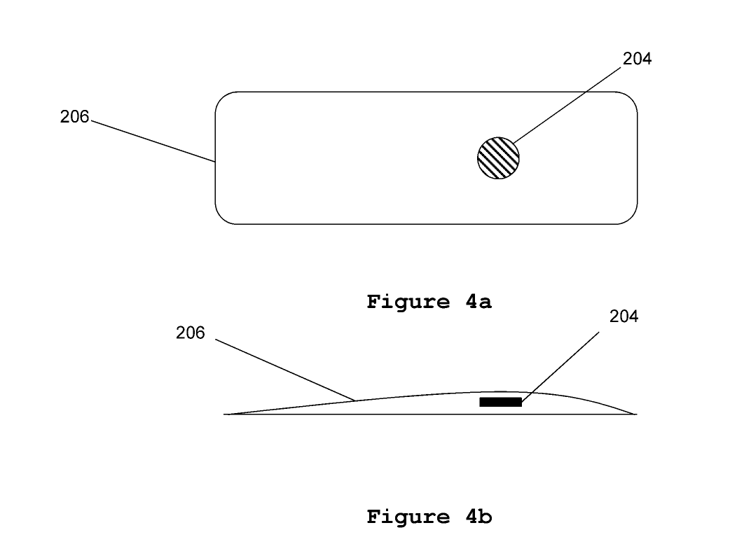

The flexible portion 206 with the first element 204 are shown in further detail in FIG. 4. In particular, FIG. 4(a) shows a top view of the flexible portion 206 with the first element 204 and FIG. 4(b) shows a side view of the flexible portion 206 with the first element 204.

In this particular example, the flexible portion 206 is substantially flat and has a substantially rectangular cross-sectional shape. The rectangular shape has curved corners such that when the flexible portion 206 with the first element is positioned within the eye of a patient, surgical trauma of surrounding tissue can be minimised. A person skilled in the art will appreciate that other suitable shapes of the flexible portion 206 are envisaged.

In this example, a thickness of the flexible portion 206 tapers towards opposite ends of the flexible portion resulting in a wedge shape. The wedge shape of at least one end of the flexible portion is arranged for forming the pocket between the sclera and the choroid of the eye such that the flexible portion can be positioned in that pocket. Specifically, the tapered end is arranged to gradually open up a space between the sclera and the choroid of the eye such that a pocket is formed.

In one embodiment, the first element is located in the proximity of an edge of the flexible portion. However, other positions within the flexible portion are envisaged.

The first and the second elements 202, 204 may have one or more magnetic materials, which may have different shapes. For example, the magnetic material may have a circular cross-sectional shape, a rectangular cross-sectional shape, a ring-like cross-sectional shape, or any other shape suitable shape. The type, number and shape of the magnetic materials in the first and the second elements 202, 204 influences the magnetic forces between the first and second elements. The magnetic materials may have a magnetisation that is oriented transversally or longitudinally relative to the positioned intraocular device.

FIGS. 5 (a)-(c) illustrate the first and second elements in accordance with embodiments of the present invention in further detail. FIG. 5 (a) illustrates a second element 400 having two magnetic materials 402, 404. A corresponding configuration of the materials is implemented on a first element 406, which comprises magnetisable materials 408, 409. The second element 400 is coupled to an intraocular device 410. In this example the materials 402 and 404 are permanent magnets and the materials 408 and 409 are magnetisable materials. In a variation of the described embodiment the materials 408 and 409 may also be permanent magnets.

FIG. 5 (b) shows an example of first and second elements 414, 412 that each comprises one permanent magnetic material 418, 416 that has a rectangular shape. The permanent magnetic materials 416 and 418 have an orientation that is perpendicular to that of the permanent magnetic materials 402 and 404 shown in FIG. 5 (a).

FIG. 5 (c) shows an example of first and second elements 422, 420 that each comprise one a pair of permanent magnetic material 432, 430 and 424, 426 each having a magnetisation that is oriented along the device 410 to which the second element 420 is coupled.

FIG. 5 illustrates only a few possible configurations and a person skilled in the art will appreciate that various alternative configurations are possible.

### Example

Certain characteristics of a specific example of an intraocular system and associated method according to the present invention will now be described.

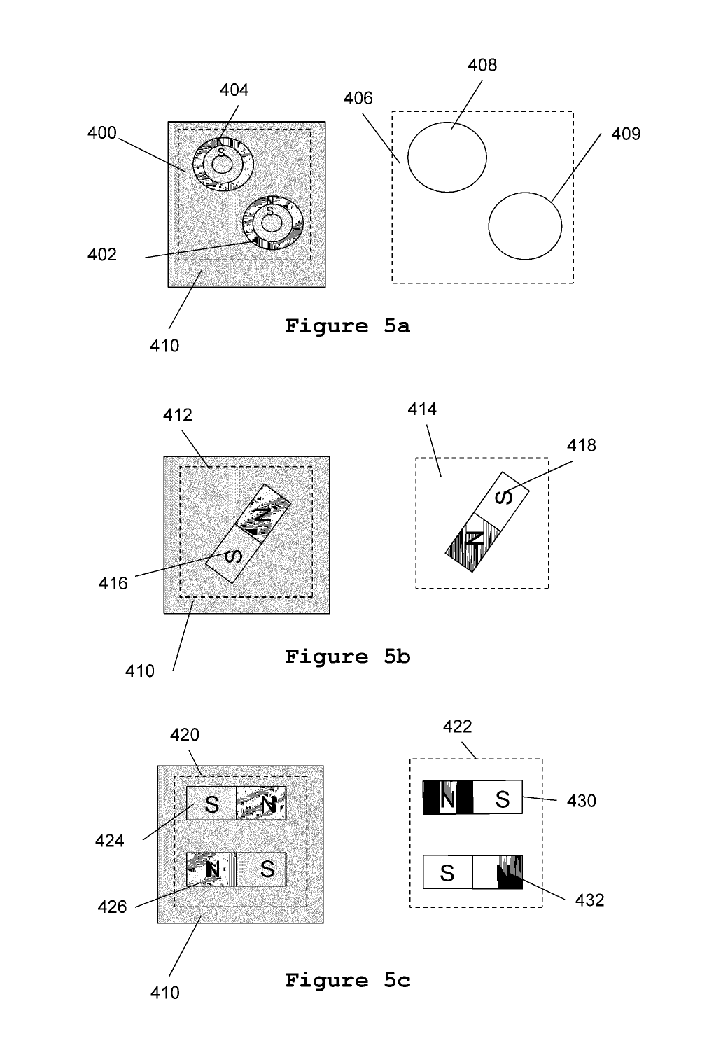

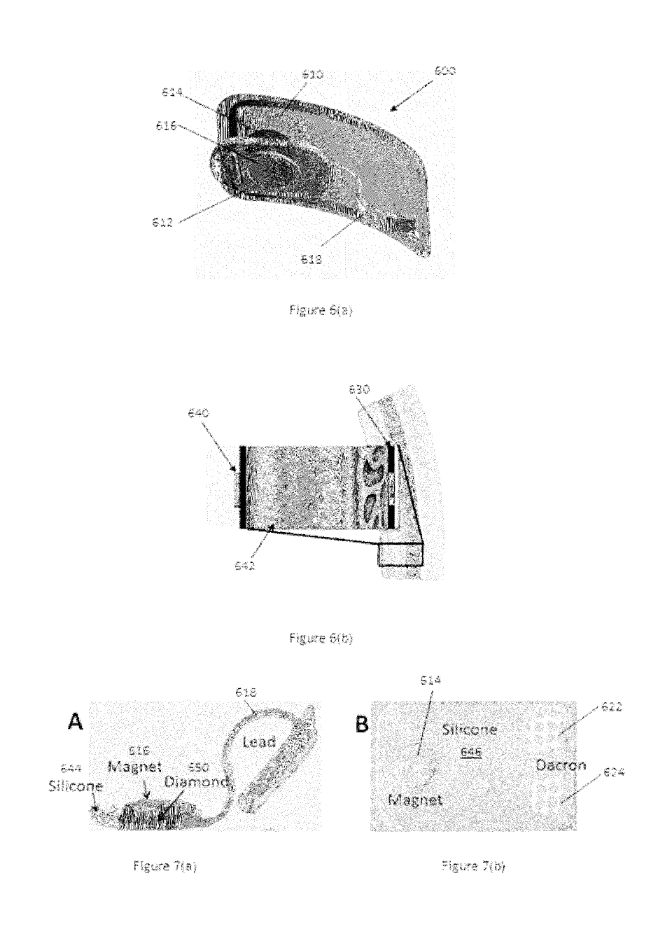

With reference to FIGS. 6a to 7b, the intraocular system 600 includes a two-component prostheses, comprising a suprachoroidal component 610 and an epiretinal component 612. FIG. 7(a) in particular shows the epiretinal component 612 and FIG. 7(b) shows the suprachoroidal component 610. A first element in the form of a backing magnet 614 is embedded in the suprachoroidal component 610, which is placed in the suprachoroidal space 630. A second element in the form of a primary magnet 616 attached to an electrode component or intraocular device, is embedded in the epiretinal component 612, to be placed at a position 640 in contact with the retina 642. In particular, the intraocular device is a diamond device 650 of approximately 800 μm thick. The suprachoroidal and epiretinal components each comprise a silicone housing 644 and 646 respectively in which their respective magnets are embedded.

The backing magnet 614 is located in the suprachoroidal space prior to epiretinal prosthesis surgery for placement of the intraocular device. In this way, the use of magnets to secure the intraocular device has less risk of misalignment compared to tacking because the positioning of the device is more precise and is predetermined by the position of the backing magnet located in the suprachoroidal space. Experimentation shows that this use of magnets is more stable than using tacks, and does not appear to cause significant migration of the device.

The system 600 comprises a 25-strand platinum lead 618 and a moulded elbow to aid in positioning the lead through a scleral wound. The suprachoroidal component 610 has two Dacron® polyethylene terephthalate patches 622 and 624 to provide mechanical stability by enabling suturing of the suprachoroidal component onto the sclera.

In this specific example, in use, the distance from the suprachoroidal backing magnet 614 to the epiretinal primary magnet 616 is typically 0.9-1.3 mm (due to the thickness of the epiretinal component 612 and the tissue between the two components).

During experimentation, rare earth neodymium-iron-boron (NdFeB) magnet discs with a diameter of 3 mm and thickness of 0.3 mm were selected and tested to determine the force between magnet pairs over a range of anatomically-relevant separation distances. The magnetic flux density was measured using a Gaussmeter and the separation was determined using 3D printed acrylonitrile butadiene styrene spacers to ensure that measurements were repeatable and precise. The force measurement between magnet pairs was calculated by setting a weight to one of a pair of coupled magnets to induce decoupling under gravitational force. In order to weaken the magnetic force between magnet pairs to put less strain on the retina once implanted, individual magnets were heat treated. This was done by placing them on a digital hotplate and measuring the magnetic flux density on the Gaussmeter after exposure to set temperature points for approximately 1 minute (25° C.-150° C.).

In addition, re-testing these magnets 1 week post heat-treatment indicated that the drop in the magnet strength under this temperature technique is permanent. After heating the magnet to 100° C., the measured magnetic flux density has decreased by 50%, where 100% represents the combined magnetic flux density of two unmodified magnets. Further, all NdFeB magnets irrespective of whether they have been heat-treated have negligible coupling strength when separated by more than 2.5 mm.

Through experimentation it was found that variations in the retina-choroidal tissue thickness will have a limited effect on the tissue force as the force exerted on the tissue is limited to less than 5 mN at 50% and 1 mN at 10%. It is believed that the force of the unmodified magnets (100%) is too high for use in such as delicate position as upon the retina and thus the heat-modified magnets may be necessary to provide a lower force. It is contemplated that the magnetic force may need to be reduced to at least 50% of its original strength.

The magnets were also coated with biocompatible films, such as thin metallic or polymer coatings. This is because non-biocompatible elements in NdFeB magnets make them unsuitable for in vivo implantation; for example, Nd readily corrodes in chloride rich environments. Various types of coating were tested, including titanium and gold coatings applied using electron-beam evaporation or sputter coating, and parylene C coating with a thickness of around 8 μm. Only the parylene coating was found to be suitable in protecting the underlying NdFeB magnet.

Experimentation was also done to study the in vivo characteristics of the system 600. The key criteria for successful implementation of the system 600 were twofold: (i) capacity to position and secure the device and (ii) avoid trauma to the retina. To determine whether the magnets met the two key criteria, short-term implantation was observed at two ends of the force spectrum: strong magnet pairing (75% of original strength, corresponding to around 38-55 mN) and weak magnet pairings (10-20%, corresponding to around 5-15 mN). When the force used was 5-15 mN, the apparent impression between the epiretinal component 614 and the tissue was not as evident, which suggests that this lower force range may be tolerable by the elasticity of the tissue.

The contour of the silicone housing of the components 610 and 612 may also be modified to optimise the pressure and force distribution in order to reduce the risk of vascular and/or tissue damage induced by pressure points.

It is further proposed that a high acuity diamond electrode system will be later coupled with the magnets in the active device. The electrode diameters in this system may have an electrode diameter of 125 μm². The separation between the electrodes and the tissue may be significantly smaller than 125 μm. Moreover, the position of the diamond electrodes relative to the underlying retina is significant to the working of the system 600. The component 612 ought to be placed as close as possible to area centralis of the eye, as this is the area where the ganglion cells are tightly packed and thus misalignment of epiretinal component 612 can result on the unwanted stimulation of retinal ganglion cell axons leading to imprecise or unexpected percepts. Experimentation suggests that the magnetic coupling between the two components 610 and 612 place the electrodes relatively close to the target retinal tissue. The distance between the electrodes and the underlying retina may be less than 50 μm providing a better outcome than that reported by groups using tacks.

Although the invention has been described with reference to particular examples, it will be appreciated by those skilled in the art that the invention may be embodied in many other forms.

What is claimed is:

1. A method of positioning an intraocular device at an intraocular position, the method comprising the steps of:
providing a first and a second element, the first and the second element being selected such that a magnetic force attracts the first element and the second element to each other;
positioning the first element in a suprachoroidal space of an eye;
positioning the intraocular device in an intraocular space at a portion of tissue of the eye; and
positioning the second element in the intraocular space of the eye;
wherein the first element, the second element and the intraocular device are positioned such that the portion of the tissue of the eye and at least a portion of the intraocular device are positioned between the first and second elements such that the magnetic force at least contributes to securing the intraocular device at the portion of the tissue of the eye.

2. The method of claim 1 wherein the portion of the tissue of the eye comprises a portion of the retina and wherein the intraocular device is a retinal prosthesis.

3. The method of claim 1 wherein the second element is coupled to the intraocular device and is surround by, or embedded in, a portion of the intraocular device.

4. The method of claim 1 comprising coupling the intraocular device and the second element to each other.

5. The method of claim 1 wherein the first element is coupled to or embedded in a flexible portion that is shaped to facilitate insertion and positioning of the flexible portion with the second element in the suprachoroidal space of the eye.

6. The method of claim 1 comprising coupling the first element to a flexible portion such that the first element is attached to the flexible portion or partially surrounded by the flexible portion, the flexible portion being shaped to facilitate insertion and positioning of the flexible portion with the second element in the suprachoroidal space of the eye.

7. The method of claim 6 comprising embedding the first element in the flexible portion.

8. The method of claim 5 wherein the flexible portion comprises a tapered end-portion that facilitates insertion of the flexible portion into the suprachoroidal space of the eye.

9. The method of claim 5 wherein the flexible portion comprises a biocompatible polymeric material that is sufficiently flexible to adapt conform to a curvature of suprachoroidal space.

10. The method of claim 1 wherein the first and second elements are selected and positioned such that the intraocular device is secured at a desired position without the need for additional fasteners.

11. The method of claim 1 wherein the first and second elements are arranged to position the intraocular device in a desired position and the method comprises securing the intraocular device subsequently in the desired position using suitable fasteners such as tacks.

12. The method of claim 1 comprising positioning the intraocular device in a predetermined angular orientation relative to the retina using the magnetic force.

13. The method of claim 12 wherein the first and second elements comprise materials that have magnetic properties that are arranged such that the magnetic force is directed to position the first and second elements in the predetermined angular orientation relative to each other.

14. The method of claim 1 comprising forming an incision through the sclera of the eye such that the first element or a flexible portion comprising the first element can be inserted into the suprachoroidal space.

15. The method of claim 1 comprising moving the sclera and the choroid from each other by inserting the flexible portion with the first element into the suprachoroidal space.

16. The method of claim 13, comprising heat treating at least one of the first and second elements prior to positioning the first and second elements, in order to reduce a magnetic strength between the first and second elements from an original strength prior to heat treatment.

17. An intraocular system, comprising:
an intraocular device for positioning in an intraocular space of an eye;
a flexible portion arranged for locating in the suprachoroidal space of the eye;
a first element coupled to, or surrounded by, the flexible portion;
a second element arranged for positioning in the intraocular space of the eye;
wherein the first element and the second element are arranged such that a magnetic force attracts the first element and the second element to each other, and wherein the first element, the second element and the intraocular device are arranged such that, when the intraocular device is positioned, a portion of tissue of the eye and at least a portion of the intraocular device are positioned between the first and second elements such that the magnetic force at least contributes to securing the intraocular device at the tissue of the eye.

18. The intraocular system of claim 17 wherein the flexible portion comprises a biocompatible polymeric material that is sufficiently flexible to conform to a curvature of suprachoroidal space.

19. The intraocular system of claim 18 wherein the flexible portion comprises a tapered end-portion that facilitates insertion of the flexible portion into the suprachoroidal space of the eye.

20. The intraocular system of claim 17 wherein the first element is embedded in the flexible portion.

21. The intraocular system of claim 17 wherein at least one of the first and the second elements comprises a permanent magnetic material.

22. The intraocular system of claim 17 wherein at least one of the first and second elements comprises one or more magnetic materials that are positioned within the first and second elements such that the magnetic force positions the first and second elements in the predetermined angular orientation relative to each other.

23. The intraocular system of claim 17 wherein the intraocular device is a retinal prosthesis.
